(19) United States
(12) Patent Application Publication
(10) Pub. No.: US 2022/0220480 A1
(43) Pub. Date: Jul. 14, 2022

(54) METHODS AND COMPOSITIONS FOR TREATMENT OF NLRP3 INFLAMMASOME MEDIATED IL-1BETA DEPENDENT DISORDERS

(71) Applicants: INSERM (INSTITUT NATIONAL DE LA SANTÉ ET DE LA RECHERCHE MÉDICALE), Paris (FR); CENTRE HOPITALIER UNIVERSITAIRE DE NICE, Nice (FR); UNIVERSITE COTE D'AZUR, NICE (FR)

(72) Inventors: Laurent BOYER, NICE CEDEX 3 (FR); Océane DUFIES, NICE CEDEX 3 (FR); Anne DOYE, NICE CEDEX 3 (FR); Johan-Victor COURJON, NICE CEDEX 3 (FR); Céline LOUBATIER, NICE CEDEX 3 (FR); Grégory MICHEL, NICE CEDEX 3 (FR); Cédric TORRE, NICE CEDEX 3 (FR); Orane VISVIKIS, NICE CEDEX 3 (FR); Patrick MUNRO, NICE CEDEX 3 (FR)

(21) Appl. No.: 17/603,997

(22) PCT Filed: Apr. 16, 2020
(86) PCT No.: PCT/EP2020/060701

§ 371 Date: Oct. 15, 2021

(30) Foreign Application Priority Data
Apr. 17, 2019 (EP) 19305502.7

(51) Int. Cl. C12N 15/113; A61K 31/519; A61K 31/5377; A61P 31/04; C12N 9/12

(52) U.S. Cl. CPC C12N 15/1137; A61K 31/519; C12Y 207/11001; A61P 31/04; C12N 9/12; A61K 31/5377

(57) ABSTRACT

The present invention relates to a PAK-1 and/or PAK-2 inhibitor for use in the treatment of NLRP3 inflammasome mediated IL-1beta dependent disorders in a subject in need thereof. Inventors invalidated PAK-1 and/or PAK-2 in BMDM by transfecting siRNA targeting either PAK-1 and/or PAK-2 (PAK-3 is predominantly expressed in the brain). After 72 hours of siRNA expression, cells were stimulated by LPS and the CNF1 toxin for 8 hours. They observed that cells invalidated for PAK-1 had a reduced Caspase-1 activation compared to the control cells or cells invalidated for PAK-2. Similar results were observed when the IL-1β maturation was monitored. Confirming this data, the use of PAK-1 inhibitors (IPA-3 and FRAX597) were sufficient to block the IL-1β maturation observed in macrophages treated with LPS and CNF1 as well as Caspase-1 activation measured using FAM-FLICA.

### FIELD OF THE INVENTION

The invention is in the field of inflammatory disorders. More particularly, the invention relates to methods and compositions for treatment of NLRP3 inflammasome mediated IL-1beta dependent disorders.

### BACKGROUND OF THE INVENTION

Inflammasomes are signaling platforms assembled upon infection by cytosolic pattern recognition receptors (PRRs). The inflammasome triggered Caspase-1 activation is critical for the host defense against pathogens. During infection, NLRP3, a PRR also called Cryopyrin, triggers the assembly of an inflammasome activating Caspase-1 via the recruitment of ASC and Nek7. The NLRP3 inflammasome activation is tightly controlled both transcriptionally and post-translationally. Abnormal activation of the NLRP3 inflammasome is associated to Cryopyrin-associated syndromes (CAPS), human autoinflammatory diseases linked to NLRP3 somatic mutations activating Caspase-1 and the consecutive IL-1beta cytokine maturation. Despite the importance of the NLRP3 inflammasome regulation in inflammatory diseases, little is known about the mechanism controlling the NLRP3 activation and the upstream signaling regulating the NLRP3 inflammasome assembly.

Escherichia coli is the major cause of bacteremia and a leading cause of death with 10 million of death per year worldwide^{1,2}. Virulence factors of E. coli include the CNF1 toxin, a member of the family of RhoGTPases targeting toxins. The CNF1 toxin bears an enzymatic activity responsible for the posttranslational deamidation of a specific glutamine residue on a subset of small Rho GTPases, namely Rac, Cdc42 and RhoA³. This modification increases the flux of activated-Rho proteins and their downstream signaling pathways. Activation of small Rho GTPases by virulence factors is a common trait of various enteric and extra-intestinal Gram-negative pathogens. By modulating the host cytoskeleton, these virulence factors confer upon bacteria the property to invade epithelial cells and tissues, as well as hijack inflammatory cell responses⁴⁻⁷. Among the virulence factors family, the virulence factors targeting RhoGTPases are a family that includes more than 30 members. These virulence factors are either RhoGTPases activators or RhoGTPases inhibitors and both types have been shown to activate inflammasomes^{8,9}. The toxins inactivating RhoGTPases have been recently shown to activate the Pyrin inflammasome via the modification of the phosphorylation status of Pyrin by PKN1 and PKN2 kinases but nothing is known about the type of inflammasome that senses toxin activating RhoGTPases^{10,11}.

### SUMMARY OF THE INVENTION

The invention relates to a PAK-1 and/or PAK-2 inhibitor for use in the treatment of NLRP3 inflammasome mediated IL-1beta dependent disorders in a subject in need thereof. In particular, the invention is claimed by the claims.

### DETAILED DESCRIPTION OF THE INVENTION

By using the CNF1 toxin from E. coli as a model of, inventors investigated the sensing of the RhoGTPase activating virulence factors by inflammasomes. They provide evidence of the role of the NLRP3 inflammasome in monitoring the activity of the bacterial toxins activating the host RhoGTPases. They show that NLRP3 is activated in response to the RhoGTPases targeting toxin CNF1 from Escherichia coli. They demonstrate that this activation relies on monitoring the activity of the toxin toward the RhoGTPase Rac2. They further demonstrate that NLRP3 is activated by a signaling cascade involving the p21-activated kinases (PAK) that are necessary for IL-1β cytokine maturation. Furthermore, NLRP3 chemical inhibition or genetic invalidation diminishes the bacterial burden of E. coli expressing CNF1 during bacteremia. Altogether, their results establish NLRP3 inflammasome as bona fide receptor of effector-triggered immunity and its role during bacteremia. Their observation that the amount of Caspase-1 activation correlated with the amount of Rac2 bound to GST-PAK-RBD suggested a potential role of PAK kinases in the CNF1 triggered NLRP3 inflammasome activation. To address this point, inventors invalidated PAK-1 and/or PAK-2 in BMDM by transfecting siRNA targeting either PAK-1 and/or PAK2 (PAK-3 is predominantly expressed in the brain^{15,16}). After 72 hours of siRNA expression, cells were stimulated by LPS and the CNF1 toxin for 8 hours. They observed that cells invalidated for PAK-1 had a reduced Caspase-1 activation compared to the control cells or cells invalidated for PAK-2. Similar results were observed when the IL-1β maturation was monitored. Confirming this data, the use of PAK-1 inhibitors (IPA-3 and FRAX597) were sufficient to block the IL-1beta maturation observed in macrophages treated with LPS and CNF1 as well as Caspase-1 activation measured using FAM-FLICA.

A PAK-1 and/or PAK-2 Inhibitor for Use in the Treatment of NLRP3 Inflammasome Mediated IL-1Beta Dependent Disorders

In a first aspect, the invention relates to a PAK-1 and/or PAK-2 inhibitor for use in the treatment of NLRP3 inflammasome mediated IL-1beta dependent disorder in a subject in need thereof.

In a particular, the invention relates to a method for treating NLRP3 inflammasome mediated IL-1beta dependent disorder in a subject in need thereof comprising a step of administering said subject with a therapeutically effective amount of a PAK-1 and/or PAK-2 inhibitor.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein “NLRP3 inflammasome mediated IL-1beta dependent disorder” refers to disorder which is related to diseases linked to NLRP3 abnormal activation and the consecutive IL-1β cytokine maturation. For the first time, inventors have demonstrated that NLRP3 is activated by a signaling cascade involving the p21-activated kinases (PAK) that are necessary for IL-1β cytokine maturation. Furthermore, inventors have observed that the use of PAK-1 inhibitors (IPA-3 and FRAX597) were sufficient to block the IL-1β maturation observed in macrophages treated with LPS and CNF1.

Accordingly, the NLRP3 inflammasome mediated IL-1beta dependent disorder, wherein said disorder is selected from the group consisting of: autoimmune disease; age-related macular degeneration (AMD), autoinflammatory diseases; inflammatory responses, inflammatory skin diseases, psoriasis and dermatitis (for example, atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); lupus nephritis (LN); diabetes mellitus (e.g. Type I diabetes mellitus or insulin dependent diabetes mellitis); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia; Cryopyrin-associated periodic syndromes (CAPS) Alzheimer disease, Atherosclerosis, Myocardial infarction, Asthma and allergic airway inflammation, Gout, Nonalcoholic fatty liver disease and nonalcoholic steatohepatitis, Multiple sclerosis or experimental autoimmune encephalitis, Oxalate-induced nephropathy, Hyperinflammation following influenza infection, Stroke, Silicosis, Myelodysplastic syndrome, Contact hypersensitivity, Traumatic brain injury.

In a particular embodiment, the NLRP3 inflammasome mediated IL-1beta dependent disorder is psoriasis. Psoriasis is characterized by discrete areas of skin inflammation with redness, thickening, intense scaling, and in some cases, itching. The disease has significant impact on the quality of life of affected individuals, both physically and psychologically. Today there is no cure for psoriasis, and treatment is directed at reducing the severity and extent of the psoriatic plaques and the related symptoms.

As used herein, the term “subject” refers to any mammals, such as a rodent, a feline, a canine, and a primate. In a particular embodiment, the subject is human. Particularly, in the present invention, the subject has or is susceptible to have NLRP3 inflammasome mediated IL-1beta dependent disorder. In a particular embodiment, the subject has or is susceptible to have psoriasis. In another embodiment, the psoriasis is induced by anti-PD1/anti-PD-L1 treatment.

As used herein, the term “NLRP3” refers to Nucleotide-binding oligomerization domain-like receptor including a pyrin domain 3. Nucleotide-binding oligomerization domain-like receptors (“NLRs”) include a family of intracellular receptors that detects pathogen-associated molecular patterns (“PAMPs”) and endogenous signal danger molecules.

NLRPs represent a subfamily of NLRB that include a Pyrin domain and are constituted by proteins such as NLRP1, NLRP3, NLRP4, NLRP6, NLRP7, and NLRP12. NLRPs are involved in the formation of multiprotein complexes termed inflammasomes. The NLRP3 inflammasome forms a molecular platform inside macrophages and microglial cells, catalyzing the activation of the protease Caspase-1. Caspase-1 is responsible for converting the potent pro-inflammatory cytokine interleukin-1 beta (IL-1 β) from an inactive to an active secreted form.

As used herein the term “IL-1beta” has its general meaning in the art and refers to Interleukin-1 beta. IL-1 beta is a member of the Interleukin 1 cytokine family. This cytokine is produced as a proprotein, which is proteolytically processed to its active form by Caspase 1 (CASP1/ICE). This cytokine is an important mediator of the inflammatory response, and is involved in a variety of cellular activities, including cell proliferation, differentiation, and apoptosis.

As used herein, the term “PAK-1” has its general meaning in the art and refers to P21-Activated Kinase 1, also known as Serine/threonine-protein kinase PAK-1, or P21 protein (Cdc42/Rac)-activated kinase 1. PAK-1 is a member of p21-activated kinases family (PAKs) involved in the ERK activation, MAPK pathway activation and that are critical effectors that link the Rho GTPases to cytoskeleton reorganization and nuclear signalling and have been implicated in a wide range of biological activities.

As used herein, the term “PAK-1 inhibitor” refers to any compound that is able to inhibit the activity or expression of PAK-1. In particular the PAK-1 inhibitor blocks PAK-1 interaction with proteins involved in ERK pathway and MAPK pathway such as RAF-1 (CRAF), inhibits its phosphorylation, or blocks MAPK cascade. The term “PAK-1 antagonist” refers to a compound that selectively blocks or inactivates PAK-1. As used herein, the term “selectively blocks or inactivates” refers to a compound that preferentially binds to and blocks or inactivates PAK-1 with a greater affinity and potency, respectively, than its interaction with the other sub-types or isoforms of the PAKs family.

Example of PAK-1 inhibitors include the compounds described in WO2004007504, WO2006072831, WO2007023382, WO2007072153, WO2009086204, WO2010071846, WO2011044264, WO2011044535, WO2011156640, WO2011156646, WO2011156775, WO2011156780, WO2011156786, and WO 2013026914.

Additional examples of PAK-1 inhibitors include, but are not limited to, staurosporine, 3-hydroxystaurosporine, K252a, CEP-1347, OSU-03012, DW12, FL172 (disclosed in Yi et al., Biochemical Pharmacology, 2010, 80:683-689, the disclosure of which with respect to PAK-1 inhibitor compounds is hereby incorporated herein by reference), IPA3 (commercially available from Tocris), PF-3758309, PAK10 (available from Calbiochem), EKB569, TKI258, FRAX-597 (available from Tocris) and SU-14813. In some embodiments, the PAK-1 inhibitor is a macrocyclic lactone. As used herein, the term “macrocyclic lactones” has its general meaning in the art and refers to macrocyclic lactones and macrocyclic lactones derivatives described in Lespine A. Lipid-like properties and pharmacology of the anthelmintic macrocyclic lactones. Expert Opin Drug Metab Toxicol. 2013 December; 9(12): 1581-95. Macrocyclic lactones, like ivermectin, are capable of inhibiting PAK-1 activity (e.g. HASMIMOTO ET AL: “Ivermectin inactivates the kinase PAK-1 and blocks the PAK-1 dependent growth of human ovarian cancer and NF2 tumor cell lines”, DRUG DISCOV. THERAPEUTICS, vol. 3, no. 6, 2009, -2009, pages 243-246). Examples of macrocyclic lactones include those described in WO 2012078605, WO 2012150543, WO2011075592, W0199316189, and WO2012028556. In some embodiments, examples of macrocyclic lactones include but are not limited to Ivermectin (Stromectol), Doramectin, Selamectin, Moxidectin, Milbemycin, Abamectin, Nemadectin and Eprinomectin. In a particular embodiment, the inhibitor of PAK-1 is AZ13711265. AZ13711265 is well known in the art, its CAS number is 2016806-55-4 and has the following chemical formula and structure in the art C28H35FN6O3S:

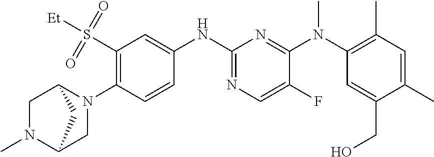

In some embodiments, the PAK-1 inhibitor is an inhibitor of PAK-1 expression. An “inhibitor of expression” refers to a natural or synthetic compound that has a biological effect to inhibit the expression of a gene. In a particular embodiment of the invention, the inhibitor of gene expression is a siRNA, an antisense oligonucleotide or a ribozyme. For example, anti-sense oligonucleotides, including anti-sense RNA molecules and anti-sense DNA molecules, would act to directly block the translation of PAK-1 mRNA by binding thereto and thus preventing protein translation or increasing mRNA degradation, thus decreasing the level of PAK-1, and thus activity, in a cell. For example, antisense oligonucleotides of at least about 15 bases and complementary to unique regions of the mRNA transcript sequence encoding PAK-1 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. PAK-1 gene expression can be reduced by contacting a subject or cell with a small double stranded R A (dsPvNA), or a vector or construct causing the production of a small double stranded R A, such that PAK-1 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing PAK-1. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor consists in a vector that comprises the CRISPR/cas 9 protein and the appropriate RNA guide for disrupting the expression level of the gene encoding for PAK-1. In some embodiments, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

As used herein the terms “administering” or “administration” refer to the act of injecting or otherwise physically delivering a substance as it exists outside the body (e.g., PAK1 and/or PAK-2 inhibitor) into the subject, such as by oral, mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof. In a particular embodiment, a topical administration is performed to the subject. More particularly, the PAK-1 and/or PAK-2 inhibitors are formulated as a cream for a topical administration. In another embodiment, an oral administration is performed to the subject. In a further embodiment, intravenous administration is performed to the subject.

By a “therapeutically effective amount” is meant a sufficient amount of a PAK1 and/or PAK2 inhibitor for use in a method for the treatment of NLRP3 inflammasome mediated il-1beta dependent disorder at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic 20 adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Combined Preparation:

The PAK-1 and/or PAK-2 inhibitor as described above is combined with an inhibitor of NLRP3 inflammasome.

The PAK-1 and/or PAK-2 inhibitor as described above is also combined with a classical treatment.

Accordingly, in a second aspect, the invention relates to i) a PAK-1 and/or PAK-2 inhibitor and ii) an inhibitor of NLRP3 inflammasome used as a combined preparation for treating NLRP3 inflammasome mediated IL-1beta dependent disorder in a subject.

Accordingly, the invention relates to i) a PAK-1 and/or PAK-2 inhibitor and ii) a classical treatment used as a combined preparation for treating NLRP3 inflammasome mediated IL-1beta dependent disorder in a subject.

As used herein, the terms “combined treatment”, “combined therapy” or “therapy combination” refer to a treatment that uses more than one medication. The combined therapy may be dual therapy or bi-therapy.

In a particular embodiment, i) a PAK-1 and/or PAK-2 inhibitor and ii) an inhibitor of NLRP3 inflammasome as a combined preparation according to the invention for simultaneous, separate or sequential use in the treatment of NLRP3 inflammasome mediated IL-1beta dependent disorder.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

As used herein, the term “an inhibitor of NLRP3 inflammasome” refers to an inhibitor which inhibits the recruitment of the adapter protein the apoptosis-associated speck-like (ASC) pro-caspase-1 leading to caspase-1 production and subsequent interlukin-1∘ (IL-1β) maturation and release.

In a particular embodiment, the inhibitor of NLRP3 inflammasome is MCC950. MCC950 blocks the release of IL-1beta induced by NLRP3 activators, such as ATP, MSU and nigericin, by preventing oligomerization of the inflammasome adaptor protein ASC (apoptosis-associated speck-like protein containing CARD). MCC950 is well known in the art and has the cas number 210826-40-7 and chemical formula: C20H24N2O5S.

In a particular embodiment, the inhibitor of NLRP3 inflammasome is described in the following patent applications: WO2017/129897; WO2013/007763; WO2016/12322; WO2017/031161; WO2017/017469; WO2017/184746; WO2019/025467; WO2019/034693.

In a particular embodiment, the inhibitor of NLRP3 inflammasome is selected from the group consisting of: a sufonylurea drug such as glyburide, including functionally equivalent derivatives thereof, for example, glyburide precursors or derivatives that lack the cyclohexylurea moiety, or functionally equivalent precursors or derivatives that contain the sulfonyl and benamido groups. Examples include 5-chloro-2-methoxy-N-[2-(4-sulfamoylphenyl)-ethyl]-benzamide and 1-[(4-methylbenzene)sulfonyl]-1 H-1,3-benzodiazol-2-amine. Functionally equivalent precursors or derivatives of glyburide include precursors or derivatives that retain the activity of glyburide, at least in part, to inhibit or reduce the activity of NLRP3 inflammasome, e.g. retain at least about 25% of the activity of glyburide, preferably about 50% of glyburide activity, for example, at least about 70%, 80%, or 90% if glyburide activity.

In another embodiment, the invention relates to i) a PAK-1 and/or PAK-2 inhibitor and ii) Caspase-1 inhibitor used as a combined preparation for treating NLRP3 inflammasome mediated IL-1beta dependent disorder in a subject.

In another embodiment, the inhibitor is a Caspase-1 inhibitor. The Caspase-1 inhibitor may be a direct inhibitor of Caspase-1 enzymatic activity, or may be an indirect inhibitor that inhibits initiation of inflammasome assembly or inflammasome signal propagation. Caspase-1 inhibitors for use in the present invention may be antioxidants, including reactive oxygen species (ROS) inhibitors. Examples of such Caspase-1 inhibitors include, but are not limited to, flavonoids including flavones such as apigenin, luteolin, and diosmin; flavonols such as myricetin, fisetin and quercetin; flavanols and polymers thereof such as catechin, gallocatechin, epicatechin, epigallocatechin, epigallocatechin-3-gallate and theaflavin; isoflavone phytoestrogens; and stilbenoids such as resveratrol. Also included are phenolic acids and their esters such as gallic acid and salicyclic acid; terpenoids or isoprenoids such as andrographolide and parthenolide; vitamins such as vitamins A, C and E; vitamin cofactors such as co-enzyme Q10, manganese and iodide, other organic antioxidants such as citric acid, oxalic acid, phytic acid and alpha-lipoic acid, and Rhus verniciflua stokes extract. The Caspase-1 inhibitor may be a combination of these compounds, for example, a combination of a-lipoic acid, co-enzyme Q10 and vitamin E, or a combination of a Caspase 1 inhibitor(s) with another inflammasome inhibitor such as glyburide or a functionally equivalent precursor or derivative thereof. The Caspase-1 inhibitor may be a small molecule inhibitor, as one of skill in the art will appreciate. Non-limiting examples include cyanopropanate-containing molecules such as (S)-3-((S)-1-((S)-2-(4-amino-3-chlorobenzamido)-3,3-dimethylbutanoyl)pyrrolidine-2-carboxamido)-3-cyano-propanoic acid, as well as other small molecule caspase-1 inhibitors such as (S)-1-((S)-2-{[1-(4-amino-3-chloro-phenyl)-methanoyl]-amino}-3,3-dimethyl-butanoyl)-pyrrolidine-2-carboxylic acid ((2R,3 S)-2-ethoxy-5-oxo-tetrahydro-furan-3-yl)-amide. Such inhibitors may be chemically synthesized.

NLRP3 inflammasome or Caspase-1 may also be inhibited using immunological inhibitors such as monoclonal antibodies prepared using the well-established hybridoma technology developed by Kohler and Milstein (Nature 256, 495-497(1975)). Hybridoma cells can be screened immunochemically for production of antibodies specifically reactive with a selected NLRP3 or caspase-1 protein region, the full amino acid sequences of which is known in the art and provided herein and the monoclonal antibodies can be isolated. The term “antibody” as used herein is intended to include fragments thereof which also specifically react with a NLRP3 or caspase-1 protein according to the invention, as well as chimeric antibody derivatives, i.e., antibody molecules resulting from the combination of a variable non-human animal peptide region and a constant human peptide region. The inflammasome inhibitor may also be an oligonucleotide inhibitor using, for example, anti-sense or RNA interference inhibitors such as siRNA. NLRP3- or caspase-1-encoding nucleic acid molecules, such as that provided herein, may be used to prepare oligonucleotide inhibitors effective to bind to NLRP3 or caspase-1 nucleic acid and inhibit the expression thereof. The term “antisense oligonucleotide” as used herein means a nucleotide sequence that is complementary to at least a portion of a target NLRP3 or caspase-1 nucleic acid sequence. The term “oligonucleotide” refers to an oligomer or polymer of nucleotide or nucleoside monomers consisting of naturally occurring bases, sugars, and intersugar (backbone) linkages. The antisense oligonucleotides of the present invention may be ribonucleic or deoxyribonucleic acids and may contain naturally occurring bases including adenine, guanine, cytosine, thymidine and uracil, or modified bases such as xanthine, hypoxanthine, 2-aminoadenine, 6-methyl, 2-propyl and other alkyl adenines, 5-halo uracil, 5-halo cytosine, 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydrodyl guanine and other 8-substituted guanines, other aza and deaza uracils, thymidines, cytosines, adenines, or guanines, 5-tri-fluoromethyl uracil and 5-trifluoro cytosine. Antisense oligonucleotides of the invention may also contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates and phosphorodithioates. In addition, the antisense oligonucleotides may contain a combination of linkages, for example, phosphorothioate bonds may link only the four to six 3′-terminal bases, may link all the nucleotides or may link only 1 pair of bases.

In a particular embodiment, i) a PAK-1 and/or PAK-2 inhibitor and ii) a classical treatment as a combined preparation according to the invention for simultaneous, separate or sequential use in the treatment of NLRP3 inflammasome mediated IL-1beta dependent disorder.

In a particular embodiment, the classical treatment refers to immunosuppressive corticosteroids or non-steroidal therapies; immunotherapy: recombinant human IL-1β receptor antagonist; neutralizing monoclonal anti-IL-1β antibody; immune checkpoint inhibitors.

In a particular embodiment the invention relates to i) a PAK-1 and/or PAK-2 inhibitor and ii) a corticosteroid used as a combined preparation for treating NLRP3 inflammasome mediated IL-1beta dependent disorder in a subject.

In a particular embodiment, i) a PAK-1 and/or PAK-2 inhibitor and ii) a corticosteroid as a combined preparation according to the invention for simultaneous, separate or sequential use in the treatment of NLRP3 inflammasome mediated IL-1beta dependent disorder.

As used herein, the term “corticosteroid” is well known in the art and refers to class of steroid hormones that are produced in the adrenal cortex as well as the synthetic analogues of these hormones. Two types of classes of corticosteroid exist in the art: glucocorticoids and mineralocorticoids. The corticosteroid for use in the invention is selected from the group consisting of: Flugestone (flurogestone); Fluorometholone; Medrysone; Prebediolone acetate; chlormadinone acetate, cyproterone acetate, medrogestone, medroxyprogesterone acetate, megestrol acetate, and segesterone acetate; Chloroprednisone; Cloprednol; Difluprednate; Fludrocortisone; Fluocinolone; Fluperolone; Fluprednisolone; Loteprednol; Methylprednisolone; Prednicarbate; Prednisolone; Prednisone; Tixocortol; Triamcinolone; Alclometasone; Beclometasone; Betamethasone; Clobetasol; Clobetasone; Clocortolone; Desoximetasone; Dexamethasone; Diflorasone; Difluocortolone; Fluclorolone; Flumetasone; Fluocortin; Fluocortolone; Fluprednidene; Fluticasone; Fluticasone furoate; Halometasone; Meprednisone; Mometasone; Mometasone furoate; Paramethasone; Prednylidene; Rimexolone; Ulobetasol (halobetasol); Amcinonide; Budesonide; Ciclesonide; Deflazacort; Desonide; Formocortal (fluoroformylone); Fluclorolone acetonide (flucloronide); Fludroxycortide (flurandrenolone, flurandrenolide); Flunisolide; Fluocinolone acetonide; Fluocinonide; Halcinonide; Triamcinolone acetonide; Cortivazol; RU-28362.

In a particular embodiment the invention relates to i) a PAK-1 and/or PAK-2 inhibitor and ii) a nonsteroidal drug used as a combined preparation for treating NLRP3 inflammasome mediated IL-1beta dependent disorder in a subject.

In a particular embodiment, i) a PAK-1 and/or PAK-2 inhibitor and ii) a nonsteroidal drug as a combined preparation according to the invention for simultaneous, separate or sequential use in the treatment of NLRP3 inflammasome mediated IL-1beta dependent disorder.

As used herein, the term “nonsteroidal drug” refers to a class of drugs which decrease inflammation. The nonsteroidal drug for use in the invention is selected from the group consisting of: Aspirin (acetylsalicylic acid); Diflunisal (Dolobid); Salicylic acid and other salicylates Salsalate (Disalcid); Ibuprofen; Dexibuprofen; Naproxen; Fenoprofen; Ketoprofen; Dexketoprofen; Flurbiprofen; Oxaprozin; Loxoprofen; Indomethacin; Tolmetin; Sulindac; Etodolac; Ketorolac; Diclofenac; Aceclofenac; Nabumetone; Piroxicam; Meloxicam; Tenoxicam; Droxicam; Lornoxicam; Phenylbutazone; Mefenamic acid; Meclofenamic acid; Flufenamic acid; Tolfenamic acid; Celecoxib; Clonixin.

As used herein, the term “immunotherapy” has its general meaning in the art and refers to the treatment that consists in administering an immunogenic agent i.e. an agent capable of inducing, enhancing, suppressing or otherwise modifying an immune response.

In another embodiment the invention relates to i) a PAK-1 and/or PAK-2 inhibitor and ii) a neutralizing monoclonal anti-IL-1β antibody used as a combined preparation for treating NLRP3 inflammasome mediated IL-1beta dependent disorder in a subject.

In a particular embodiment, i) a PAK-1 and/or PAK-2 inhibitor and ii) a neutralizing monoclonal anti-IL-1β antibody as a combined preparation according to the invention for simultaneous, separate or sequential use in the treatment of NLRP3 inflammasome mediated IL-1beta dependent disorder.

As used herein, the term “a neutralizing monoclonal anti-IL-1β antibody” refers to an antibody that blocks or reduces at least one activity of a polypeptide comprising the epitope to which the antibody specifically binds. The neutralizing antibody reduces IL-1β biological activity in in cellulo and/or in vivo tests. In the context of the invention, the neutralizing monoclonal anti-IL-1β antibody is canakinumab (trade name Ilaris, developed by Novartis).

In another embodiment the invention relates to i) a PAK-1 and/or PAK-2 inhibitor and ii) a recombinant human IL-1β receptor antagonist used as a combined preparation for treating NLRP3 inflammasome mediated IL-1beta dependent disorder in a subject.

In a particular embodiment, i) a PAK-1 and/or PAK-2 inhibitor and ii) a recombinant human IL-1β receptor antagonist as a combined preparation according to the invention for simultaneous, separate or sequential use in the treatment of NLRP3 inflammasome mediated IL-1beta dependent disorder. In the context of the invention, the recombinant human IL-1B receptor antagonist is Anakinra (marketed as Kineret® by Swedish Orphan Biovitru).

In another embodiment the invention relates to i) a PAK-1 and/or PAK-2 inhibitor and ii) an immune checkpoint inhibitor used as a combined preparation for treating NLRP3 inflammasome mediated IL-1beta dependent disorder in a subject.

In a particular embodiment, i) a PAK-1 and/or PAK-2 inhibitor and ii) an immune checkpoint inhibitor as a combined preparation according to the invention for simultaneous, separate or sequential use in the treatment of NLRP3 inflammasome mediated IL-1beta dependent disorder.

As used herein, the term “immune checkpoint inhibitor” has its general meaning in the art and refers to any compound inhibiting the function of an immune inhibitory checkpoint protein.

As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist.

In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.S70, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1 MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

In some embodiments, CTLA-4 (Cytotoxic T-Lymphocyte Antigen-4) antagonists are selected from the group consisting of anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), Tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097; 5,855,887; 6,051,227; and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. A preferred clinical CTLA-4 antibody is human monoclonal antibody (also referred to as MDX-010 and Ipilimumab with CAS No. 477202-00-9 and available from Medarex, Inc., Bloomsbury, N.J.) is disclosed in WO 01/14424. With regard to CTLA-4 antagonist (antibodies), these are known and include Tremelimumab (CP-675,206) and Ipilimumab.

In some embodiments, the immunotherapy consists in administering to the subject a combination of a CTLA-4 antagonist and a PD-1 antagonist.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM-3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Ga19). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an IDO inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

Pharmaceutical Composition:

The PAK-1 and/or PAK-2 inhibitor for use according to the invention alone and/or combined with NLRP3 inhibitor and classical treatment as described above may be combined with pharmaceutically acceptable excipients, and optionally sustained-release matrices, such as biodegradable polymers, to form pharmaceutical compositions.

Accordingly, in a further aspect, the invention relates to a pharmaceutical composition comprising a PAK-1 and/or PAK-2 inhibitor for treating NLRP3 inflammasome mediated IL-1beta dependent disorder.

In a particular embodiment, the pharmaceutical composition according the invention, wherein the PAK-1 and/or PAK-2 inhibitor is FRAX597.

In a particular embodiment, the pharmaceutical composition according the invention, wherein the PAK-1 and/or PAK-2 inhibitor is IPA3.

In a particular embodiment, the pharmaceutical composition according the invention, wherein the PAK-1 and/or PAK-2 inhibitor is AZ13711265.

In a particular embodiment, the pharmaceutical composition according the invention comprising i) a PAK-1 and/or PAK-2 inhibitor and ii) an inhibitor of NLRP3.

In a particular embodiment, the pharmaceutical composition according the invention comprising i) a PAK-1 and/or PAK-2 inhibitor and ii) a classical treatment.

In a particular embodiment, the pharmaceutical composition according the invention comprising i) a PAK-1 and/or PAK-2 inhibitor and ii) a neutralizing monoclonal anti-IL-1β antibody.

In a particular embodiment, the pharmaceutical composition according the invention comprising i) a PAK-1 and/or PAK-2 inhibitor and ii) a recombinant human IL-1β receptor antagonist.

In a particular embodiment, the pharmaceutical composition according the invention comprising i) a PAK-1 and/or PAK-2 inhibitor and ii) an immune checkpoint inhibitor.

In a particular embodiment, the pharmaceutical composition according the invention comprising i) a PAK-1 and/or PAK-2 inhibitor and ii) a corticosteroid.

In a particular embodiment, the pharmaceutical composition according the invention comprising i) a PAK-1 and/or PAK-2 inhibitor and ii) a nonsteroidal drug.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In a particular embodiment, the pharmaceutical composition (PAK-1 and/or PAK-2 inhibitor alone or combination with NLRP3 inhibitor, and classical treatment) is formulated as a cream. Said pharmaceutical composition is suitable for a topical administration. In another embodiment, the pharmaceutical composition (PAK-1 and/or PAK-2 inhibitor alone or combination with NLRP3 inhibitor, or classical treatment) is formulated for an oral administration. In a further embodiment, the pharmaceutical composition (PAK-1 and/or PAK-2 inhibitor alone or combination with NLRP3 inhibitor, or classical treatment) is formulated for an intravenous administration.

Method for Screening:

In a further aspect, the invention relates to a method of screening a drug suitable for the treating NLRP3 inflammasome mediated IL-1beta dependent disorder comprising i) providing a test compound and ii) determining the ability of said test compound to inhibit the expression or activity of PAK-1 and/or PAK-2.

Any biological assay well known in the art could be suitable for determining the ability of the test compound to inhibit the activity or expression of PAK-1 and/or PAK-2. In some embodiments, the assay first comprises determining the ability of the test compound to bind to PAK-1 and/or PAK-2. In some embodiments, a population of cells then contacted and activated so as to determine the ability of the test compound to inhibit the activity or expression of PAK-1 and/or PAK-2. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to inhibit a biological activity or expression. It is to be understood that test compounds capable of inhibiting the activity or expression of PAK-1 and/or PAK-2, as determined using in vitro methods described herein, are likely to exhibit similar modulatory capacity in applications in vivo. Typically, the test compound is selected from the group consisting of peptides, petptidomimetics, small organic molecules, antibodies (e.g. intraantibodies), aptamers or nucleic acids. For example the test compound according to the invention may be selected from a library of compounds previously synthesised, or a library of compounds for which the structure is determined in a database, or from a library of compounds that have been synthesised de novo.

The invention will be further illustrated by the following figures and examples. However, these examples and figures should not be interpreted in any way as limiting the scope of the present invention.

### FIGURES

FIG. 1: CNF1-triggered IL-1β maturation requires NLRP3. (a) BMDMs isolated from BALB/c mice were transfected with the indicated siRNA for 72 h prior to 6 h of CNF1 treatment (500 ng/mL). The active Caspase 1 was detected using the FAM-FLICA probe. Cells harboring FAM-FLICA dots were counted positive using the Fiji Software (n=1500-2000 cells per condition). (b) Quantification of FAM-FLICA positive cells in WT (grey) or NLRP3 KO BMDMs (black) (n=1000 cells per condition). p-value: ****<0.0001, compared to control BMDMs (c) WT or NLRP3 KO BMDMs were treated with CNF1 (500 ng/mL), LPS (100 ng/mL) or both for 8 h prior to supernatant and cell lysates collection and immunoblot analysis. (d) BMDMs extracted from BALB/c mice were transfected with siRNA targeting the indicated isoform of RacGTPase for 72 h and treated with CNF1 (500 ng/mL) and LPS (100 ng/mL) for 8 h. Supernatant and cell lysates were analyzed by immunoblot. (e) iBMDM were transfected with the indicated siRNA for 72 h before to be treated or not with CNF1 (500 ng/mL) for 6 h prior to be analyzed using a GST-Pak Effector pull down assay. The Rac2 associated to the GST-Pak-RBD beads is indicated as Rac2-GTP.

FIG. 2: Rac2-NLRP3 Signaling Depends on Pak1 Kinase

(a-b) BMDMs isolated from BALB/c mice were transfected for 72 h with siRNA targeting either NLRP3, Nek7, Pak1, Pak2 as indicated and non-targeting siRNA were used as control. Cells were either treated with vehicle or CNF1 (500 ng/mL) and LPS (100 ng/mL) for 8 h or treated with 1 μM MCC950 for 45 min as indicated. Supernatants and cell lysates were analyzed by immunoblots (c-d) BMDMs isolated from BALB/c mice were transfected with Pak1 and Pak2 targeting siRNA or with non-targeting siRNA for 72 h and treated either with 1 μM MCC950, 5 μM IPA-3, 1 μM FRAX597 or vehicle for 45 min prior treatment with CNF1 (500 ng/mL) and LPS (100 ng/mL) treatment for 8 h. (c) Supernatants and cell lysates were analyzed by immunoblot. (d) BMDMs isolated from BALB/c mice were treated with vehicle (Control) or treated either with 1 μM MCC950, 5 μM IPA-3, 1 μM FRAX597 for 45 min prior treatment with CNF1 (500 ng/mL) for 8 as indicated. Active Caspase 1 was stained with FAM-FLICA, analyzed by microscopy and FAM-FLICA positive cells were counted. (n=1000 per conditions). p-value ****<0.0001, compared to the CNF1-treated BMDMs (e) HEK293T cells were transfected with plasmids encoding NLRP3 inflammasome component (myc-NLRP3, ASC-GFP, mCaspase-1) together with Flag-Rac2Q61E (Q61E), myc-Pak1 wild type (WT) or myc-Pak1T423E (T423E) and caspase-1 cleavage was analyzed by immunoblot.

FIG. 3: NLRP3 inflammasome controls bacteremia triggered by E. coli expressing CNF1. (a-b) Female mice were intravenously infected with 107 E. coli expressing CNF1 (E. coli CNF1+), prior to the collection of peripheral blood at 4, 24 and 48 h for the measurement of bacteremia (a) BALB/c mice were injected intraperitoneally with 50 mg/kg MCC950 once a day (n=10 per group) (b) WT or NLRP3 KO C57BL/6j mice were analyzed (n=6 per group). Data are expressed as the geometric mean±95 CI *p<0.05.

FIG. 4: (a-c) BMDMs extracted from C57BL/6J mice knocked out for the indicated inflammasome were treated or not with LPS (100 ng/mL) and CNF1 (500 ng/mL) for 8 h. Supernatants and cell lysates were collected and (a) protein were analyzed by immunoblot or (b) mRNA levels analyzed using qRT-PCR, IL-1β was normalized using the 36B4 housekeeping gene. (c) mRNA levels of NLRP3, NLRC4 and mefv (PYRIN) were measure using qRT-PCR and normalized using the 36B4 housekeeping gene.

FIG. 5: 8 weeks old C57BL/6J mice were shaved two days before the experiment. One day before administration of imiquimod and daily for 5 days, mice were intraperitoneally injected with vehicle (60% PEG, 40% PBS) or with the Pak1 inhibitor AZ13711265 (10 mg/kg). Mice were treated in the back daily for 4 days either with Vaseline or with Aldara cream corresponding to 3,125 mg of imiquimod (IMQ) to induce psoriasis-like phenotype. The erythema and scaling was analyzed by an adapted version of the clinical PASI score, 0: none; 1: slight; 2: moderated; 3: strong; 4: very strong phenotype. The total score is the cumulative score of scaling and erythema. Results are expressed as mean±SEM. p-value: *<0.01, ***<0.001, compared to control

### EXAMPLE

Material & Methods

Ethics Statement

This study was carried out in strict accordance with the guidelines of the Council of the European Union (Directive 86/609/EEC) regarding the protection of animals used for experimental and other scientific purposes. The protocol was approved by the Institutional Animal Care and Use Committee on the Ethics of Animal Experiments of Nice, France (reference: NCE/2012-64).

Bacterial Strains and Toxins

The E. coli UTI89 clinical isolate was originally obtained from a patient with cystitis¹⁹ and the UTI89 CNF1+ streptomycin resistant strain generation and culture condition were previously described⁸. For the infections, a 1/100 dilution of an overnight culture was inoculated and grown in up to OD600=1.2 using a Luria-Bertani (LB) medium supplemented with streptomycin (200 μg/ml). Bacteria were harvested by centrifugation and washed twice in PBS before dilution in PBS to obtain the desired bacterial concentrations for the mouse infection experiments. Recombinant wild-type Cytotoxic Necrotizing Factor-1 (CNF1) and its catalytically inactive form (CNF1-C866S) were produced and purified as previously reported 20. The recombinant proteins were passed through a polymyxin B column (AffinityPAK Detoxi-Gel, Pierce). The endotoxins removal was verified using a colorimetric LAL assay (LAL QCL-1000, Cambrex). Each stock of the CNF I preparation (2 mg/ml) was shown to contain less than 0.5 endotoxin units/ml.

Cell Culture, Transfection and Inhibitors

HEK 293T cells were obtained from ATCC (CRL-3216) and maintained according to ATCC instructions. Bone-marrow derived macrophages (BMDMs) were extracted from femurs of 6-10 weeks-old BALB/c, C57BL/6J or C57BL/6J KO mice as indicated in the figure legends and were cultured in RPMI GlutaMax medium (Life Technologies) supplemented with 100 ng/mL M-CSF (Premium grade, Miltenyi Biotec), 10% heat-inactivated FBS (Biowest) and 50 μg/mL gentamycin (Life Technologies) at 37° C. in an atmosphere containing 5% CO2. The cells were seeded at a concentration of 106 cells per well in a 6-well plate. After 6 days of differentiation, BMDMs were used for experiments. NLRP3, NLRC4, PYRIN, and GSDMD knock-out BMDMs were a kind gift from M. Lamkanfi. HEK 293T cells were transfected with plasmids using Lipofectamine 2000 (Life technologies) according to the manufacturer's instructions. SiRNAs were transfected in HEK 293T cells or BMDMs during 72 h using Lipofectamine RNAiMAX Reagent (ThermoFisher Scientific) according to the manufacturer instructions. Cells were transfected as indicated in figure legends with siRNAs targeting NLRP3 (L-053455-00, Dharmacon), Rac1 (L-041170-00, Dharmacon), Rac2 (L-041171-01, Dharmacon), Nek7 (J-063266-09, Dharmacon), PAK1 (L-048101-00, Dharmacon), PAK2 (L-040615-00, Dharmacon) or non-targeting control siRNA (D-001810-10, Dharmacon). BMDMs were pre-treated with inhibitors for 45 minutes: 1 μM CP-456773 ou MCC950 (Sigma), Z-VAD, 10 μM Y-27632 (Sigma), 5 μM IPA3 (Tocris), 1 μM FRAX597 (Tocris) in 2% FBS containing RPMI followed by the addition of CNF1 500 ng/mL and/or ultrapure LPS 100 ng/mL (Invivogen) as indicated in the figure legends. Cells pre-treated with ultrapure LPS for 6 h followed by the addition of Nigericin 504 (Invivogen) or ATP 5 mM (Invivogen) for 30 minutes were used as positive control of NLRP3 inflammasome activation.

Mouse Model of Infection

Female BALB/c from Janvier (Le Genest St Isle, France) were injected intraperitoneally with MCC950 50 mg/kg/24 h. Mice were injected intravenously with 107 CFUs of E. coli as previously described⁸. For the determination of bacteremia, blood was collected from the tail vein at the indicated times post-infection, serially diluted in sterile PBS and plated on LB plates containing streptomycin (200 μg/ml) and the plates were incubated for 16 h at 37° C. Injection quality was controlled by plating blood samples obtained from the mice at 5 min after injection. NLRP3 knock-out mice were kindly provided by V. Petrilli and have been previously described²¹. Female NLRP3 KO and female congenic WT C57BL/6J littermate mice were injected intravenously with 107 CFUs of E. coli and the determination of bacteremia was monitored as previously described⁸.

Reconstituted NLRP3 Inflammasome in HEK293T Cells System

HEK293T cells were transfected with plasmids encoding the NLRP3 inflammasome components as previously described¹⁴. HEK293T cells were transfected with plasmids encoding myc-NLRP3, ASC-GFP, mPro-Caspase1, and pro-IL-1β-Flag and Rac2, the constitutively active mutant of Rac2 mimicking CNF1-induced deamidation Rac2Q61E or Rac2T17N a dominant negative mutant of Rac2 for 16 h. The monitoring of Caspase-1 or IL-1β cleavage was performed using supernatant immunoblotting.

In Vitro Kinase Assay

500 ng of recombinant purified PAK1 were incubated with 1 μg of recombinant human NLRP3 protein (abcam, ab165022), and with 50 μM ATP and 4 μCi of [γ 32 P]-ATP in kinase buffer (50 mM HEPES pH 7.3, 50 mM NaCl, 0.05% Triton X-100, 10 mM β-glycerophosphate, 5 mM NaF, 10 mM MgCl2, and 0.2 mM MnCl2) at 30° C. for 30 min in a final volume of 39 μl. Reaction was stopped by adding 15 ul of LDS (ThermoFisher Scientific) and 6 ul of DTT 500 mM. 20 ul of samples were analyzed by electrophoresis using Bolt 4-12% Bis-Tris Plus gels (ThermoFisher Scientific) followed by Coomassie blue staining and autoradiography.

Immunofluorescence Staining and Antibodies

Caspase 1 activation was detected using the fluorescent probe FAM-FLICA (ImmunoChemistry Technologies) after 6 h of treatment, according to the manufacturer instructions. After labelling, cells were fixed in 4% paraformaldehyde for 15 min and blocked with 2% TBS-BSA. Cells were incubated with mouse anti-NLRP3 antibody (clone Cryo-2, Adipogen) for 1 hour followed by incubation with secondary antibody TexasRed anti-mouse IgG (TI-2000, Vector Laboratories) and Hoechst (Thermo Fisher Scientific) for 30 minutes. Cells were imaged using Nikon MR confocal microscope. Antibodies used in this study were: rabbit anti-IL-1β (GTX74034, Genetex), mouse anti-Caspase-1 (clone Casper-1, Adipogen), mouse anti-Rac (clone 102/Rac1, BD Biosciences), goat anti-Rac2 (ab2244, Abcam), mouse anti-NLRP3 (clone Cryo-2, Adipogen), rabbit anti-Nek7 (ab133514, Abcam), rabbit anti-PAK1 (2602, CST), rabbit anti-PAK2 (2608, CST), rabbit anti-MLKL (28640, CST), rabbit anti-Gasdermin D (209845, Abcam), mouse anti-β-actin (AC-74, Sigma), mouse anti-Myc (9E10, Roche), mouse anti-HA (16B12, Covance), mouse anti-Flag (clone M2, Sigma), mouse anti-GFP (clone 7.1, 13.1, Roche).

Immunoprecipitation

BMDMs from BALB/c mice were plated at 5.106 cells in a 10 cm cell culture dish. After 6 days of differentiation, cells were treated with CNF1 (500 ng/mL), LPS (100 ng/mL) or both for 6 h. Cells were washed with cold PBS and lysed with 1 mL of cold Nonidet P-40 lysis buffer (50 mM Tris pH7.5, 150 mM NaCl, 10 mM MgCl2, 1% Nonidet P-40) for 30 min at 4° C. Cell lysates were centrifugate at 10 000 g for 10 min at 4° C. and supernatants were incubated for 16 h at 4° C. with anti-NLRP3 antibody. Then, Dynabeads Protein G (10004D, Thermo Fisher Scientific) were added for 30 min. Dynabeads were washed three time in lysis buffer.

Results

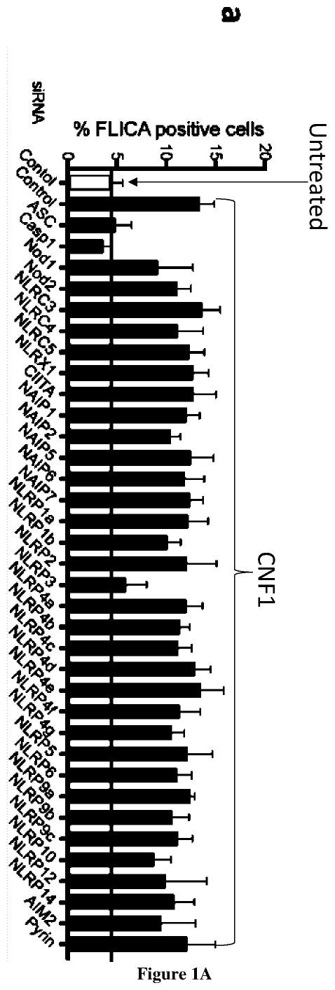

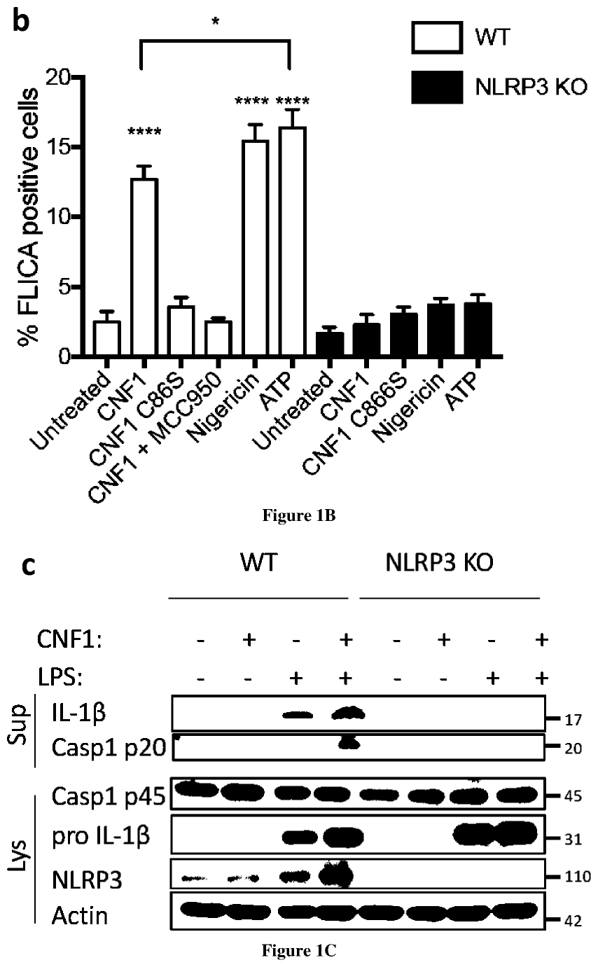

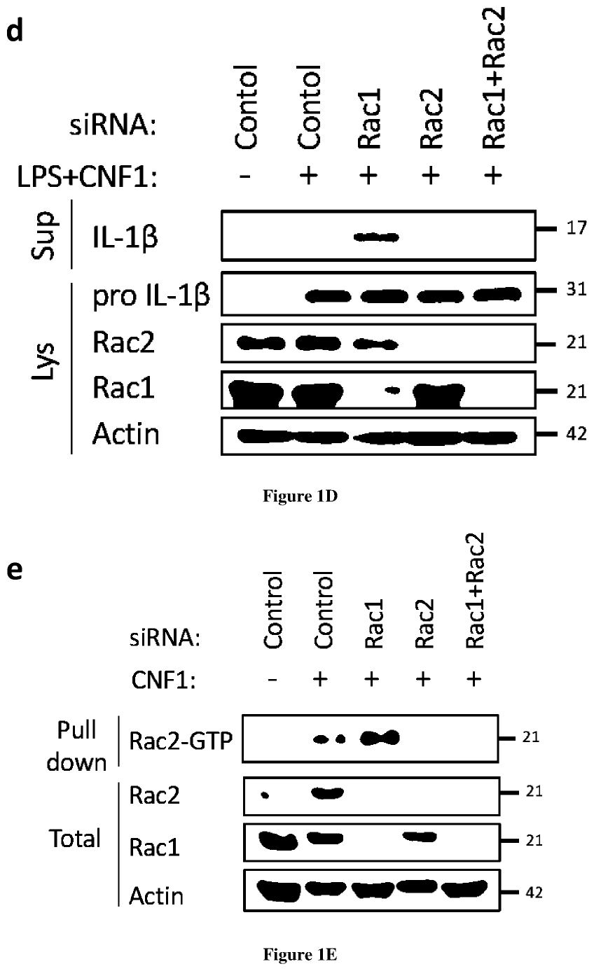

Taking advantage of the CNF1-triggered Caspase-1 activation we previously observed, we set-up an assay to monitor this activation using microscopy with a FAM-YVAD-FMK (FAM-FLICA). Primary bone marrow derived macrophages (BMDMs) were treated with CNF1 for 6 hours to trigger Caspase-1 activation. Cells were incubated with FAM-FLICA washed and fixed before to be processed for microscopy. Cells with dots of FAM-FLICA staining were determined as positive (data not shown). Next, we designed and screened a siRNA library targeting mice NLRs. To this aim, BMDMs were transfected with a siRNA library targeting mouse NLRs and Caspase-1 activation was monitored as previously described. We measured the activation of Caspase-1 triggered by the CNF1 toxin with a mean of 13% of FLICA positive cells compared to cells treated with the vehicle (4,4%) when cells were transfected with non-targeting control siRNA. We used as positive control siRNA targeting Caspase-1 and ASC that were measured with respectively a mean of 3,6% and 4,9% similar to the control not treated with CNF1 (4,4%). This result unraveled NLRP3 as the major NLR involved in the CNF1 triggered-Caspase-1 activation (FIG. 1a). We took advantage of this assay to further investigate the role of NLRP3 in the CNF1-triggered immunity. The co-treatment of the CNF1 toxin together with the NLRP3 inhibitor MCC950 (also called CP-456773) was sufficient to block the Caspase-1 activity (FIG. 1b). Similar results were found by using BMDMs isolated from NLRP3 KO mice, confirming both the involvement of NLRP3 in the CNF1-triggered immunity and the specificity of the FAM-FLICA probe to measure NLRP3 triggered Caspase-1 activity. Importantly, this assay allowed us to determine that CNF1 triggered 12.7% of FAM-FLICA positive cells, in a similar range than Nigericin or ATP treatment, respectively a mean of 15.4% and 16.4% respectively, identifying the CNF1 toxin as a bona fide NLRP3 activator (FIG. 1b). Furthermore, the amount of FAM-FLICA positive cells was dramatically reduced when BMDMs were treated with the CNF1 catalytic inactive mutant C866S with a mean of 3.6% similar to the control condition measured at 2.5%. This result provided us a first evidence of the monitoring of the CNF1 toxin activity by NLRP3 rather than the pattern of the toxin as it is classically described for Pattern Triggered Immunity (PTI)¹².

We further confirmed genetically the specific role of NLRP3 in the CNF1-triggered IL-113 maturation and Caspase-1 (FIG. 1 d and FIG. 4). The CNF1 toxin is a RhoGTPase activator and Rac2 is a hematopoietic specific RhoGTPase that was previously involved in the innate immune response to the CNF1 toxin¹³. To investigate the role of Rac2 in the NLRP3 inflammasome activation triggered by CNF1 we invalidated Rac2 and/or Rac1 by transfecting BMDM with siRNA targeting Rac2 or Rac1. Interestingly, Rac1 targeting by siRNA resulted in an increased level of IL-1β maturation triggered by the CNF1 toxin whereas targeting Rac2 was sufficient to block the CNF1-triggered IL-1β maturation (FIG. 1d). The PAK effector pull down assay is widely used to monitor the level of activated Rac GTPases in cells. Using this approach, we tested the hypothesis of an increased activated Rac2 levels when Rac1 was targeted by siRNA explaining the increased level of IL-1β maturation in CNF1 treated BMDMs. Supporting this hypothesis, the GST-PAK-RBD pull-down analysis indicated an increased activated Rac2 when Rac1 was invalidated by siRNA (FIG. 1e). Altogether these data demonstrated the critical role of Rac2 in the CNF1-triggered IL-1β maturation (FIGS. 1d and 1e).

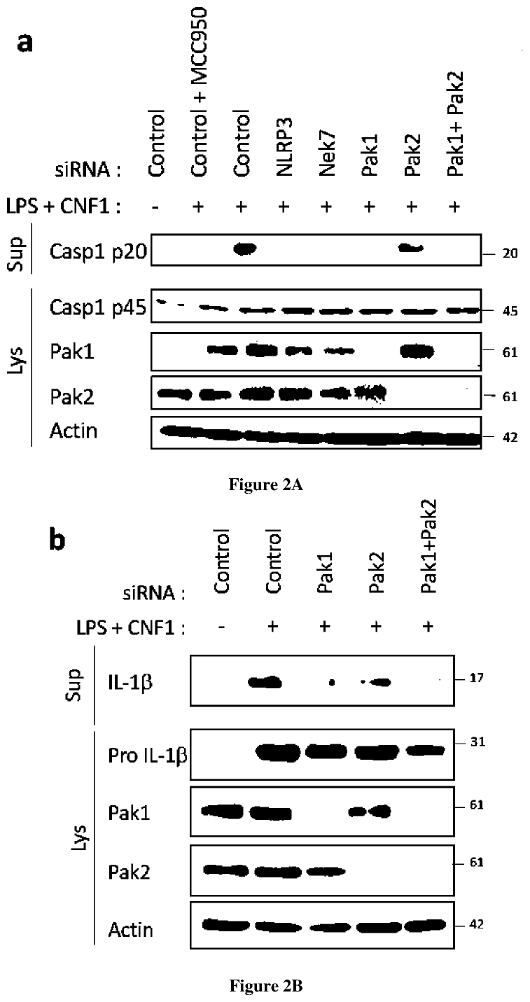

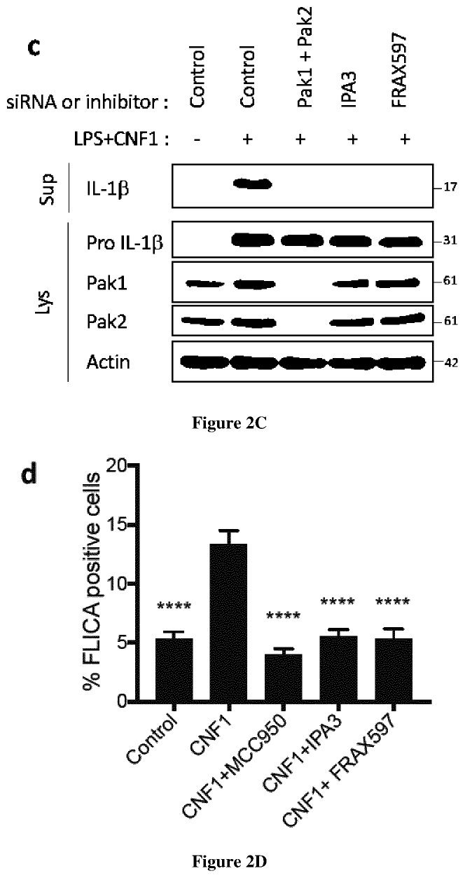

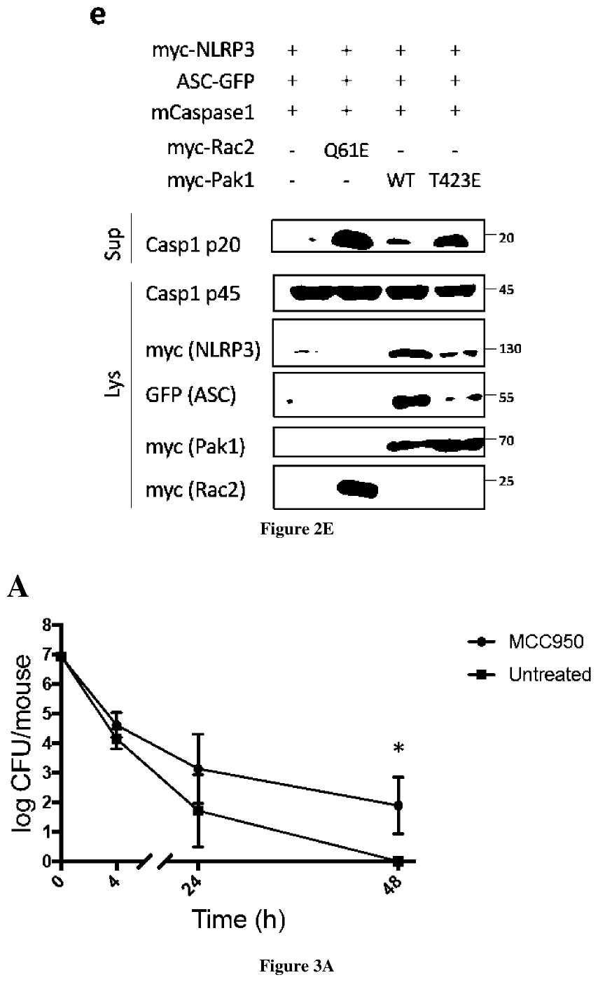

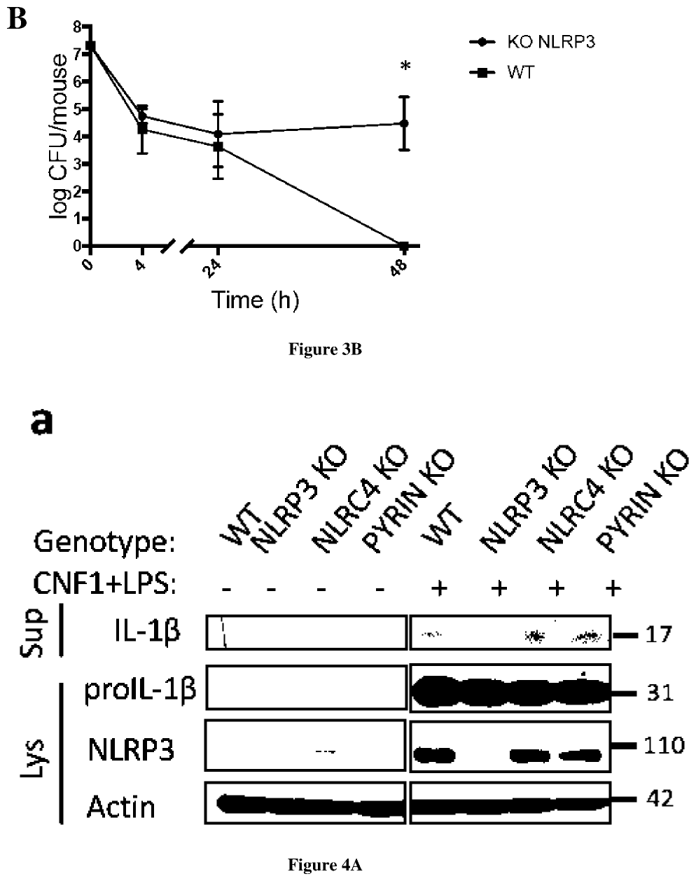

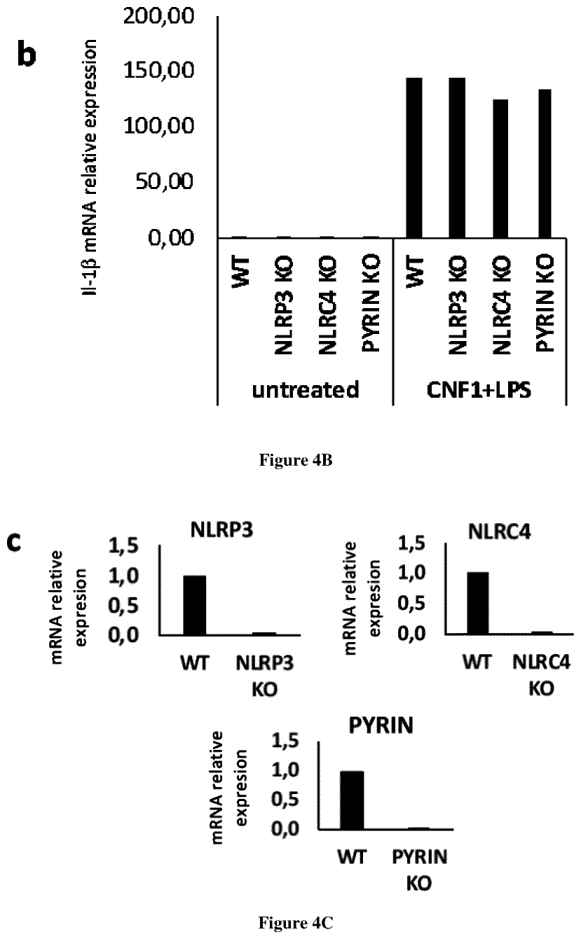

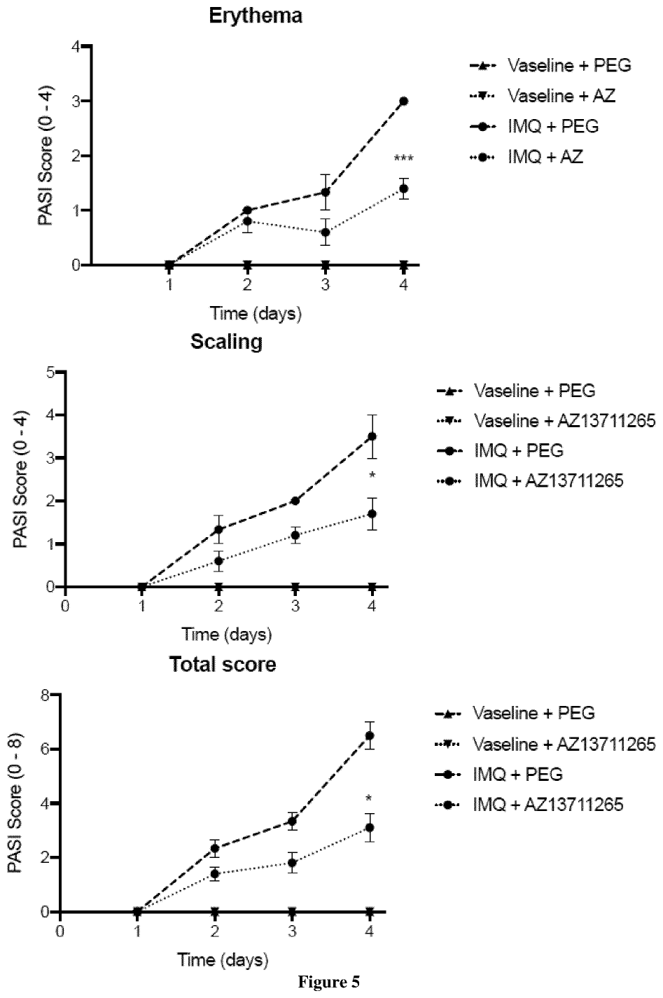

We next confirmed the biochemical association between NLRP3 and CNF1 activated-Rac2 by co-immunoprecipitation (Co-IP) experiments in BMDMs using an anti-NLRP3 antibody (data not shown). This Co-IP also revealed the association of the NLRP3 inflammasome regulator Nek7 to the Rac2-NLRP3 complex. In accordance with this observation, the downregulation of Nek7 by siRNA transfection of BMDMs inhibited the IL-1B maturation triggered by CNF1 (data not shown). In order to determine the molecular mechanism of Caspase-1 activation we took advantage of the NLRP3 inflammasome reconstitution in 293T cells by expressing murine forms of NLRP3, ASC, Nek7¹⁴. This allowed us to determine that CNF1 is sufficient to trigger the NLRP3 inflammasome activation triggered Caspase-1 cleavage and the co-treatment of CNF1 with MCC950 inhibited this Caspase-1 activation (data not shown). Taking advantage of this system, we transfected the Rac2 GTPase and various mutant forms of Rac2. We observed that constitutively active mutant forms of Rac2, ranging from the Q61E mimicking the modification made by the CNF1 toxin to the classical active mutants Q61L and G12V, were able to activate Caspase-1 in contrast to the inactive mutant Rac2T17N (data not shown). Interestingly, the strength of Caspase-1 activation observed with the activated forms of Rac2GTPases correlated with the amount of RhoGTPases bound to GST-PAK-RBD in the PAK effector pull down assay (data not shown). Altogether, these data indicated that NLRP3 senses the activation level of the RhoGTPase Rac2 proportionally to the strength of activation rather than detecting the structural modification made by the toxin as it would be predicted for a classical PRR. Our observation that the amount of Caspase-1 activation correlated with the amount of Rac2 bound to GST-PAK-RBD suggested a potential role of PAK kinases in the CNF1 triggered NLRP3 inflammasome activation. To address this point, we invalidated PAK1 and/or PAK2 in BMDM by transfecting siRNA targeting either PAK1 and/or PAK2 (PAK3 is predominantly expressed in the brain^{15,16}). After 72 hours of siRNA expression, cells were stimulated by LPS and the CNF1 toxin for 8 hours. We observed that cells invalidated for PAK1 had a reduced Caspase-1 activation compared to the control cells or cells invalidated for PAK2 (FIG. 2a). Similar results were observed when the IL-1β maturation was monitored (FIG. 2b). Confirming this data, the use of PAK1 inhibitors (IPA-3 and FRAX597) were sufficient to block the IL-1β maturation observed in macrophages treated with LPS and CNF1 (FIG. 2c) as well as Caspase-1 activation measured using FAM-FLICA (FIG. 2d). Interestingly, the IPA-3 was shown to inhibit specifically the binding of activated forms of Rac and Cdc42 to PAK1 thereby inhibiting the autophosphorylation of PAK1 in Threonine 423 whereas the FRAX597 is an ATP-competitive PAK kinase inhibitor. To further investigate the role of PAK1 in the NLRP3 inflammasome activation we took advantage of the inflammasome reconstitution system in HEK 293T cells. We expressed the activated form of Rac2 Q61E mimicking the modification made by the CNF1 toxin as a positive control for caspase-1 cleavage and compared the effect of the expression of PAK1 wild type (WT) or an activated form of PAK1 T423E. We observed that overexpression of the activated form of PAK1 was sufficient to trigger the Caspase-1 maturation (data not shown). In addition, phosphorylated forms of PAK were found co-localized in dot like structures together with NLRP3 and active caspase-1 labeled with FAM-FLICA (data not shown). PAK1 is a serine/threonine kinase and to further investigate whether NLRP3 is a substrate for PAK1 we set-up an in vitro kinase assay using PAK1 and NLRP3 recombinant proteins incubated for 20 minutes with ATPγ^{P32}. We observed the autophosphorylation of PAK1 in the condition PAK1 alone and no signal was observed in the condition with NLRP3 alone. In the condition with PAK1 and NLRP3 combined we observed the apparition of a band at the size of NLRP3 indicating the direct phosphorylation of NLRP3 by PAK1 in vitro. Altogether, our results unraveled a novel regulation of the NLRP3 inflammasome by the Rac2/PAK1 signaling axis leading to the phosphorylation of NLRP3.

We previously demonstrated that the CNF1 toxin expressed by E. coli triggered an immune response in vivo and the bacterial clearing during bacteremia⁸. To determine the importance of NLRP3 in the sensing of the CNF1 activity in vivo, we took advantage of the NLRP3 inhibitor MCC950 that was shown to be efficient in vivo¹⁷. We monitored the bacterial burden observed during mice bacteremia using control mice (vehicle) or mice injected IP with the MCC950 (50 mg/kg). Mice were infected with the same strain and concentration of CNF1 expressing E. coli and the bacteremia was measured on each infected mouse at 0 h, 4 h, 24 h and 48 h. Consistent with our previous observation, the bacterial clearing of E. coli expressing CNF1 in wild type animals was observed at 24 h and with no bacteria detectable at 48 h in all infected animals⁸. We measured a statistically significant higher bacterial load at 48 h with 56% (9/16) of animals found positive for bacteria in mice injected with the NLRP3 inhibitor compared to 0 in the control group. To genetically prove the involvement of NLRP3 in the clearing of E. coli expressing CNF1, we infected wild type mice or transgenic NLRP3 KO mice and compared the bacterial burden over the time. Consistent with the previous results obtained with the NLRP3 inhibitor, we measured no bacteria in the blood of infected WT mice while we measured a mean of 3.7 10⁴ bacteria per mouse in the blood of NLRP3 KO mice. The difference of E. coli CNF1+clearing between WT and NLRP3 KO mice was not observed when mice were infected with isogenic E. coli CNF1-strain invalidated for CNF1 (data not shown). Altogether these results unravel the critical role of NLRP3 in the clearing of CNF1 expressing bacteria and the importance of NLRP3 in the innate immune response during bacteremia. Our study shows the NLRP3 inflammasome as a major sensor of bacterial toxins activating RhoGTPases whereas PYRIN senses the RhoGTPases inactivating toxins⁹. Altogether, these studies highlight that, in parallel to the PRR detection of PAMPS, the mammalian innate immune system has evolved strategies that shares similarities with the Effector Triggered Immunity (ETI) described in plants^{12,18}, to monitor via NLRs the abnormal activation state of the RhoGTPases that are common targets of bacterial toxins and critical regulators of host cell homeostasis.

Futhermore, we investigated the effect of Pak1 kinase inhibitor on a psoriasis model. Dermal application of imiquimod induced a psoriasis-like phenotype including scaling and erythema that are scored using the PASI score. At day 4 of imiquimod administration, mice treated with imiquimod only exhibit a significant higher scaling, erythema and total PASI score (respectively 3.5, 3 and 6.5) than mice treated with imiquimod and the Pak1 inhibitor AZ13711265 (respectively 1.7, 1.4 and 3.1) (FIG. 5). These results show the effect of the Pak1 kinase inhibitor AZ13711265 in the development of imiquimod induced a psoriasis-like phenotype.

### REFERENCES

Throughout this application, various references describe the state of the art to which this invention pertains. The disclosures of these references are hereby incorporated by reference into the present disclosure.
1. Martin, G. S., Mannino, D. M., Eaton, S. & Moss, M. The epidemiology of sepsis in the United States from 1979 through 2000. N Engl J Med 348, 1546-1554 (2003).
2. Russo, T. A. & Johnson, J. R. Proposal for a new inclusive designation for extraintestinal pathogenic isolates of Escherichia coli: ExPEC. J Infect Dis 181, 1753-1754 (2000).
3. Aktories, K. & Barbieri, J. Bacterial cytotoxins: targeting eukaryotic switches. Nat Rev Micro 3, 397-410 (2005).
4. Galán, J. E. Common themes in the design and function of bacterial effectors. Cell Host Microbe 5, 571-579 (2009).
5. Bruno, V. M. et al. Salmonella Typhimurium type III secretion effectors stimulate innate immune responses in cultured epithelial cells. PLoS Pathog 5, e1000538 (2009).
6. Munro, P. et al. Activation and proteasomal degradation of rho GTPases by cytotoxic necrotizing factor-1 elicit a controlled inflammatory response. J Biol Chem 279, 35849-35857 (2004).
7. Boquet, P. & Lemichez, E. Bacterial virulence factors targeting Rho GTPases: parasitism or symbiosis? Trends Cell Biol 13, 238-246 (2003).
8. Diabate, M. et al. Escherichia coli alpha-Hemolysin Counteracts the Anti-Virulence Innate Immune Response Triggered by the Rho GTPase Activating Toxin CNF1 during Bacteremia. PLoS Pathog 11, e1004732 (2015).
9. Xu, H. et al. Innate immune sensing of bacterial modifications of Rho GTPases by the Pyrin inflammasome. Nature 513, 237-241 (2014).
10. Gao, W., Yang, J., Liu, W., Wang, Y. & Shao, F. Site-specific phosphorylation and microtubule dynamics control Pyrin inflammasome activation. Proc Natl Acad Sci USA 113, E4857-66 (2016).
11. Park, Y. H., Wood, G., Kastner, D. L. & Chae, J. J. Pyrin inflammasome activation and RhoA signaling in the autoinflammatory diseases FMF and HIDS. Nat Immunol 17, 914-921 (2016).
12. Stuart, L. M., Paquette, N. & Boyer, L. Effector-triggered versus pattern-triggered immunity: how animals sense pathogens. Nat Rev Immunol (2013).
13. Boyer, L. et al. Pathogen-derived effectors trigger protective immunity via activation of the Rac2 enzyme and the IMD or Rip kinase signaling pathway. Immunity 35, 536-549 (2011).
14. Shi, H., Murray, A. & Beutler, B. Reconstruction of the Mouse Inflammasome System in HEK293T Cells. Bio Protoc 6, (2016).
15. Manser, E., Leung, T., Salihuddin, H., Zhao, Z. S. & Lim, L. A brain serine/threonine protein kinase activated by Cdc42 and Rac1. Nature 367, 40-46 (1994).
16. Wells, C. M. & Jones, G. E. The emerging importance of group II PAKs.

Biochem J 425, 465-473 (2010).
17. Coll, R. C. et al. A small-molecule inhibitor of the NLRP3 inflammasome for the treatment of inflammatory diseases. Nat Med 21, 248-255 (2015).
18. Jones, J. D. & Dangl, J. L. The plant immune system. Nature 444, 323-329 (2006).
19. Mulvey, M. A., Schilling, J. D. & Hultgren, S. J. Establishment of a persistent Escherichia coli reservoir during the acute phase of a bladder infection. Infect Immun 69, 4572-4579 (2001).
20. Doye, A., Boyer, L., Mettouchi, A. & Lemichez, E. Ubiquitin-mediated proteasomal degradation of Rho proteins by the CNF1 toxin. Methods Enzymol 406, 447-456 (2006).
21. Martinon, F., Pétrilli, V., Mayor, A., Tardivel, A. & Tschopp, J. Gout-associated uric acid crystals activate the NALP3 inflammasome. Nature 440, 237-241 (2006).

1. A method of treating a NLRP3 inflammasome mediated IL-1beta dependent disorder in a subject in need thereof, comprising
administering to the subject a therapeutically effective amount of a PAK 1 and/or PAK2 inhibitor.

2. The method according to claim 1, wherein the PAK 1 and/or PAK2 inhibitor is siRNA.

3. The method according to claim 1, wherein the PAK 1 and/or PAK2 inhibitor is a small molecule.

4. The method according to claim 3, wherein the small molecule is IPA-3 (1,1′-Dithiodi-2-naphthtol), FRAX597 (6-[2-Chloro-4-(5-thiazolyl)phenyl]-8-ethyl-2-[[4-(4-methyl-1-piperazinyl)phenyl]amino]pyrido[2,3-d]pyrimidin-7-(8H)-one) or AZ1371126.

5. The method according to claim 1, wherein the NLRP3 inflammasome mediated IL-1beta dependent disorder is selected from the group consisting of: an infectious disease; autoimmune disease; age-related macular degeneration (AMD); autoinflammatory diseases; inflammatory responses; inflammatory skin diseases; psoriasis and dermatitis; systemic scleroderma and sclerosis; responses associated with inflammatory bowel disease; respiratory distress syndrome; dermatitis; meningitis; encephalitis; uveitis; colitis; glomerulonephritis; an allergic conditions; atherosclerosis; leukocyte adhesion deficiency; rheumatoid arthritis; systemic lupus erythematosus (SLE); lupus nephritis (LN); diabetes mellitus; multiple sclerosis; Reynaud's syndrome; autoimmune thyroiditis; allergic encephalomyelitis; Sjorgen's syndrome; juvenile onset diabetes; an immune response associated with acute and delayed hypersensitivity mediated by cytokines and T-lymphocytes; pernicious anemia (Addison's disease); diseases involving leukocyte diapedesis; central nervous system (CNS) inflammatory disorder; multiple organ injury syndrome; hemolytic anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia; Cryopyrin-associated periodic syndromes (CAPS); Alzheimer disease, Atherosclerosis, Myocardial infarction, Asthma and allergic airway inflammation, Gout, Nonalcoholic fatty liver disease, nonalcoholic steatohepatitis, Multiple sclerosis, experimental autoimmune encephalitis, Oxalate-induced, nephropathy, Stroke, Silicosis, Myelodysplastic syndrome, Contact hypersensitivity, and Traumatic brain injury.

6. The method according to claim 5, wherein the NLRP3 inflammasome mediated IL-1beta dependent disorder is psoriasis.

7. (canceled)

8. The method of claim 1, further comprising administering to the subject a neutralizing monoclonal anti-IL-1β antibody in combination with the PAK-1 and/or PAK-2 inhibitor.

9. The method of claim 1, further comprising administering to the subject a recombinant human IL-1β receptor antagonist in combination with the PAK-1 and/or PAK-2 inhibitor.

10. The method of claim 1, further comprising administering to the subject an immune checkpoint inhibitor in combination with the PAK-1 and/or PAK-2 inhibitor.

11. The method of claim 1, wherein the PAK-1 and/or PAK-2 inhibitors are formulated as a cream for a topical administration.

12. A pharmaceutical composition comprising a PAK-1 and/or PAK-2 inhibitor for treating a NLRP3 inflammasome mediated IL-1beta dependent disorder.

13. The pharmaceutical composition according to claim 12, which is formulated as a cream for topical administration.

14. A method of screening a drug suitable for treating a NLRP3 inflammasome mediated IL-1beta dependent disorder comprising i) providing a test compound and ii) determining the ability of said test compound to activate or inhibit the expression or activity of PAK-1 and/or PAK-2.

15. (canceled)

16. The method of claim 5, wherein
the infectious disease is hyperinflammation following influenza infection, sepsis or septic shock; and/or
the dermatitis is atopic dermatitis; and/or
the inflammatory bowel disease is Crohn's disease or ulcerative colitis; and/or
the allergic condition is eczema or asthma; and/or
the respiratory distress syndrome is adult respiratory distress syndrome (ARDS); and/or
the diabetes mellitus is Type I diabetes mellitus or insulin dependent diabetes mellitis; and/or
the immune response associated with acute and delayed hypersensitivity mediated by cytokines and T-lymphocytes is associated with tuberculosis, sarcoidosis, polymyositis, granulomatosis or vasculitis; and/or
the hemolytic anemia is cryoglobinemia or Coombs positive anemia.
